(19) United States
(12) Patent Application Publication
(10) Pub. No.: US 2026/0316024 A1
(43) Pub. Date: Oct. 15, 2026

(54) FOULING RESISTANT MICROFLUIDIC MIXING DEVICE FOR ROBUST MANUFACTURING OF NANOPARTICLES

(71) Applicant: THE TRUSTEES OF THE UNIVERSITY OF PENNSYLVANIA, Philadelphia, PA (US)

(72) Inventors: Daeyeon LEE, Wynnewood, PA (US); Yoon-Hu HWANG, Philadelphia, PA (US); David Aaron ISSADORE, Philadelphia, PA (US)

(21) Appl. No.: 19/485,503

(22) PCT Filed: May 17, 2024
(86) PCT No.: PCT/US2024/029998

§ 371 Date: Nov. 18, 2025

Related U.S. Application Data
(60) Provisional application No. 63/503,197, filed on May 19, 2023.

(51) Int. Cl. B01F 33/3011; A61K 47/69

(52) U.S. Cl. CPC B01F 33/3011; A61K 47/6929

(57) ABSTRACT

A microfluidic component, comprising: at least one microchannel configured to communicate a fluid flow therein, the at least one microchannel comprising at least one surface having a tethered liquid perfluorocarbon (TLP) coating disposed thereon, the at least one microchannel optionally having a cross-sectional dimension in the range of from about 10 μm to about 1000 μm. A method, comprising operating a system to formulate lipid nanoparticles, the system comprising a microfluidic component according to the present disclosure.

### CROSS-REFERENCE TO RELATED APPLICATIONS

The present application claims priority to and the benefit of U.S. patent application No. 63/503,197, “Fouling Resistant Microfluidic Mixing Device For Robust Manufacturing Of Nanoparticles” (filed May 19, 2023). All foregoing applications are incorporated herein by reference in their entireties for any and all purposes.

### TECHNICAL FIELD

The present disclosure relates to the field of microfluidics and nanoparticle production.

### BACKGROUND

Lipid nanoparticles (LNPs) are in the spotlight as delivery systems for mRNA therapeutics and have been used in the Pfizer/BioNTech and Moderna COVID-19 vaccines. However, traditional bulk mixing approaches often result in inefficient and non-uniform mixing, causing poor encapsulation efficiency and production quality. As an alternative approach, microfluidic technology can enable precision synthesis of mRNA-LNP by enabling rapid mixing and precise control of fluid flows not achievable using conventional batch processes. Moreover, the parallelization of microfluidic mixing units facilitates seamless scale-up, and simultaneously make it possible to use the same mixing unit through different phases of drug discovery and manufacturing. Despite these advantages, a major challenge in using microfluidic mixing units for LNP productions suffers from fouling and clogging of channel during rapid mixing and nanoprecipitation of LNPs, severely reducing the robustness in the operation of these devices.

### SUMMARY

In meeting the described needs, the present disclosure provides a microfluidic component, comprising: at least one microchannel configured to communicate a fluid flow therein, the at least one microchannel comprising at least one surface having a tethered liquid perfluorocarbon (TLP) coating disposed thereon, the at least one microchannel optionally having a cross-sectional dimension in the range of from about 10 μm to about 1000 μm.

Also provided is a method, comprising operating a system to formulate lipid nanoparticles, the system comprising a microfluidic component according to the present disclosure, for example according to any one of Aspects 1-12.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. Schematic for robust LNP formulation using fouling resistant device. While larger and more heterogeneous LNPs produced by (a) bulk formulation and (b) bare device are more variable in terms of RNA delivery, (c) fouling resistant device produces smaller and more homogeneous LNPs for potent RNA delivery.

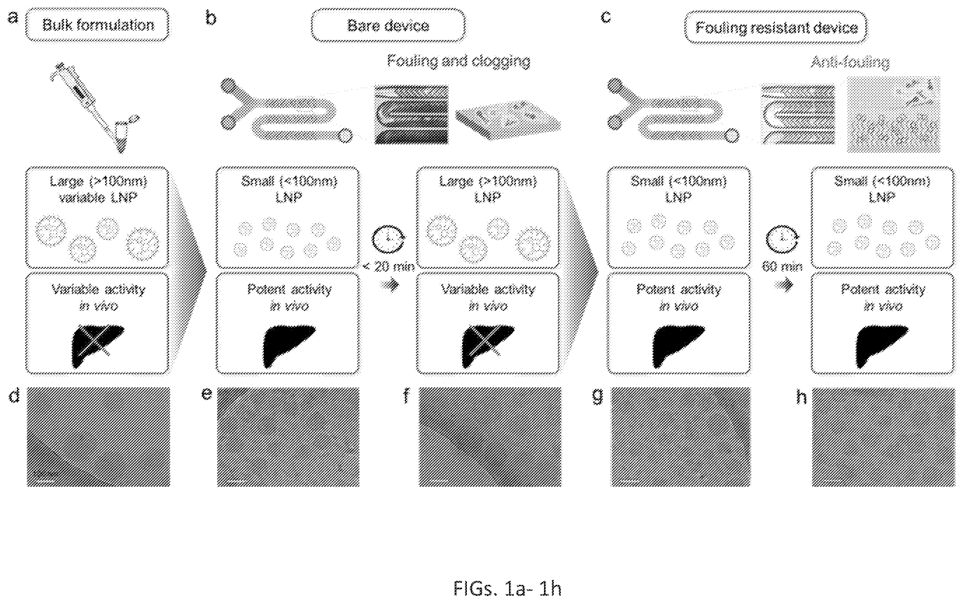

FIG. 2. (a) Schematics showing the various surface coating methods;

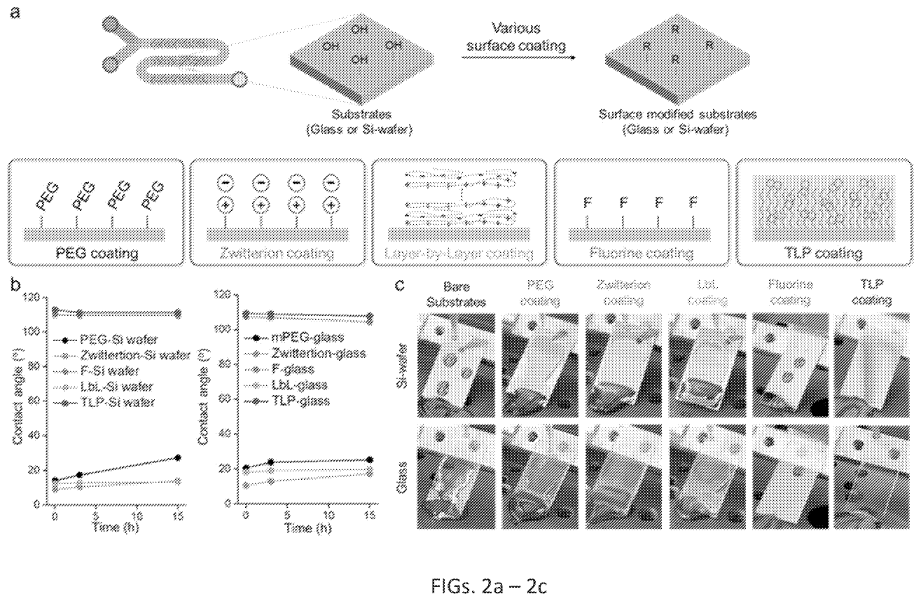

PEG, Zwitterion, layer-by-layer, fluorine, and thin layer perfluorocarbon (TLP) coatings to the Si and glass substrates (from left to right). (b) Graphs showing the contact angle analysis of water drops on the surface coated Si-wafer (left) and glass (right) substrates with various surface coating methods. (c) Photographic images showing the repelling behavior of water/EtOH (3/1) mixture solution on the surface coated Si-wafer (upper) and glass (bottom) substrates with various surface coating methods at a tilt angle of 45°.

FIG. 3. (a) Schematic illustration showing the mRNA LNP formulation using differently surface coated devices. (b) Photographic images and DLS results showing the fouling behavior and size variation of resulting mRNA LNP along the operation time, respectively. (c) PDI results of resulting mRNA LNP from TLP-coated device along the operation time. (d) mRNA encapsulation from TLP-coated device along the operation time. (e) Cryo-TEM images of mRNA LNPs produced from TLP-coated device at initial state (t=1 min, left) and final state (t=50 min, right), respectively. Scale bar: 100 nm.

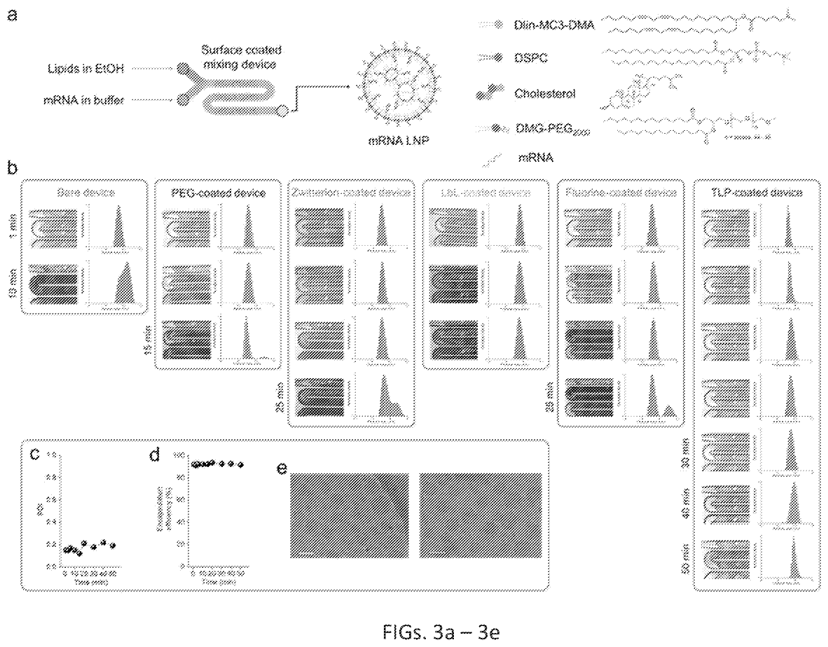

FIG. 4. (A) Schematic for robust manufacturing of RNA-LNP using SCALAR-AF chip. (B) Photograph of the SCALAR-AF chip with a staggered herringbone micromixer. Scale bar: 10 mm. (C) Schematic of the anti-fouling behavior of RNA-LNP on the lubricant layer-coated surface showing tethered perfluorocarbon (TP) bound to a substrate, which then allows a stable film of liquid perfluorodecalin (LP) to adhere to the surface. (D) Photograph showing the immiscibility between water/ethanol (3:1) mixture solution and lubricant oil phase. (E) A plot showing the maximum operating time of a microfluidic mixing chips coated different type of surface treatments. (F) Schematic and microscope images showing the fouling resistant microfluidic chip producing smaller and more homogeneous LNPs for potent RNA delivery, whereas larger and more heterogeneous LNPs are being produced by bulk formulation and conventional microfluidic chip, resulting in imprecise and unreliable delivery of RNA due to the fouling inside of devices. Scale bar: 300 μm. Cryogenic (cryo)-TEM images showing RNA-LNPs produced by bulk formulation, bare chip, and lubricant layer-coated chip before and after fouling, respectively. Scale bar: 100 nm.

FIG. 5. (A) Schematics showing various surface coating methods tested for anti-fouling behavior in microfluidic chip for manufacturing of RNA-LNPs; (i) bare, (ii) PEG, (iii) zwitterion, (vi) layer-by-layer, and (v) fluorine coatings on Si and glass substrates (from left to right). (B) Photography images showing the wetting/non-wetting of water/ethanol (3: 1) mixture solution on Si-wafer (upper) and glass (bottom) substrates with various coatings at a tilt angle of 45°. (C) Photographs (left columns) and DLS results (right columns) showing the fouling behavior and size variation of resulting RNA-LNP from each chip as a function of the operation time. Scaling bar: 150 μm.

FIG. 6. (A) Schematic of the production of RNA-LNP from TLP-coated SCALAR-AF chip. (B) Photographs showing the repellency of water/ethanol mixture (3:1) on the TLP coated Si-wafer (upper) and glass (bottom) substrates at a tilt angle of 45°, respectively. (C1-C4) Confocal micrographs showing the presence of a lubricant oil layer on the surface of the microfluidic device at different heights. (D) Micrographs revealing an excellent mixing efficiency in a 1×SCALAR-AF chip; Nile red in ethanol and FITC in water are flowed through the channel to quantify mixing at different locations along the channel; the red and green intensity profiles versus channel distance at the position of A-A′ and B-B′ (insets). Scale bar: 150 μm. (E) Quantification of mixing efficiency (±standard deviation), calculated by channel lengths required for 90% mixing for bare and 1×SCALAR-AF chip. (n=3, one-way ANOVA, ns: p>0.05) (F) Sequential micrographs showing no severe fouling during RNA-LNP formulation over a period of ~180 min. Scale bar: 150 μm. (G-I) Physical characterization of polyA LNPs produced by a 1×SCALAR-AF chip from 1 min to 180 min. (G) Hydrodynamic diameter (±standard deviation), (H) encapsulation efficiency (±standard deviation), and (I) RNA concentration (±standard deviation) of the resulting poly A LNPs from 1 min to 180 min. (n=3, one-way ANOVA, ns: p>0.05) (J) Cryo-TEM of poly A LNPs produced by 1×SCALAR-AF chip. Scale bar: 100 nm and 25 nm.

FIG. 7. (A) Photographs showing the frontside (upper) and backside (bottom) views of 256×SCALAR-AF chip. (B) Schematic of chip design summarizing the incorporation of branching geometry and ladder design architecture to uniformly distribute inputs to each mixing unit. Schematic is not to scale. (C) Circuit model of the delivery channels (R_{delivery}), resistors (Rᵣₑₛᵢₛₜₒᵣ), mixing channels (R_{mixing}), and individual mixing channel unit (R_{device}). (D) Photographs showing the experimental setup for scaling-up production of RNA-LNP from 256×SCALAR-AF chip by a custom pressure-driven flow system. (E) Photograph showing assembled aluminum holder of 256×SCALAR-AF chip, where the frontside windows for observation at different positions. (F) Photography image showing the relative throughput of 256×and 1×SCALAR-AF chips that produced LNPs for 20 min with resultant volumes of 4.8 L (256×chip; shown in 3.5 L beakers) and 10 min 0.012 L (1×chip; shown in a 15 mL falcon tube), respectively. (G) Hydrodynamic diameter (±standard deviation), (H) encapsulation efficiency (±standard deviation), and (I) RNA concentration measurements (±standard deviation) of the resulting poly A LNPs produced by the 256×SCALAR-AF chip. (n=3, one-way ANOVA, ns: p>0.05).

FIG. 8. (A) Schematic illustration showing the luciferase RNA-LNPs generated by bare chip (at t=1 min and clogged) and TLP-coated SCALAR-AF chip (at t =1 min and 60 min), respectively. (B) DLS results (±standard deviation) of luciferase RNA-LNPs produced from each device. (n=3, one-way ANOVA and Tukey's multiple comparisons, *p<0.05) (C) RNA encapsulation efficiency (±standard deviation) from each device, quantified by a RiboGreen assay. (n=3, one-way ANOVA and Tukey's multiple comparisons, *p<0.05) (D) Luciferase RNA-LNPs are formulated and administered to Hela cells at a dose of 60 ng/60,000 cells; luciferase expression is quantified by luminescence at 6 hr. (n=4-5, one-way ANOVA and Tukey's multiple comparisons, ***p<0.001) (E) Luciferase RNA-LNPs are formulated and administered to mice via tail vein injection at a dose of 4 ug per mouse. Representative IVIS imaging at 6 hr after LNP administration, showing luciferase expression in the whole body (upper) and dissected organs (bottom). (F) Quantification of luciferase signal in the whole body at 6 hr after LNP administration. (n=3-4, one-way ANOVA and Tukey's multiple comparisons, ***p<0.001; ****p<0.0001). (G) Quantification of luciferase signal in the liver. (n=3, one-way ANOVA and Tukey's multiple comparisons, **p<0.01; ***p<0.001).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments of aspects can be combined with any part or parts of any one or more other embodiments or aspects.

Lipid nanoparticles (LNPs) are in the spotlight as delivery systems for mRNA therapeutics and have been used in the Pfizer/BioNTech and Moderna COVID-19 vaccines. However, traditional bulk mixing approaches often result in inefficient and non-uniform mixing, causing poor encapsulation efficiency and production quality. As an alternative approach, microfluidic technology can enable precision synthesis of mRNA-LNP by enabling rapid mixing and precise control of fluid flows not achievable using conventional batch processes. Moreover, the parallelization of microfluidic mixing units facilitates seamless scale-up, and simultaneously make it possible to use the same mixing unit through different phases of drug discovery and manufacturing. Despite these advantages, a major challenge in using microfluidic mixing units for LNP productions suffers from fouling and clogging of channel during rapid mixing and nanoprecipitation of LNPs, severely reducing the robustness in the operation of these devices. In this study, we test various strategies that have been used for antifouling coatings for suppressing performance degradation of chaotic herringbone mixing units.

Provided here is an antifouling resistant microfluidic platform, which enables high-precision (size <100 nm, encapsulation efficiency >90%) and robust production (running time >50 min) of mRNA-LNP. We also test the biological activity of LNPs produced using the new coating strategy.

Although PDMS-based microfluidic devices offer significant advantages, they also come with some significant drawbacks, such as limited compatibility with solvents and susceptibility to deformation under high pressure. Of these drawbacks, the limited resistance to solvents is particularly concerning because PDMS swells when exposed to various organic solvents, such as chloroform and hexane, which are commonly used as co-solvents in nanoparticle synthesis. As an alternative approach, sol-gel chemistry has been employed to create a coating layer on the PDMS channel in order to improve its chemical resistance. In addition, perfluoropolyether has been used for the fabrication of microfluidic devices that exhibit resistance to solvents.

To address these challenges, we first fabricated a silicon and glass microfluidic mixing device, which have excellent solvent compatibility and compatibility with pharmaceutical manufacturing. Another concern is the fouling of the mRNA LNP constituents on the surface of the microfluidic device upon long-term operation, eventually causing the nanoparticles aggregation on the microchannel wall or channels blockages.

These fouling and clogging phenomena have been well-reported for microfluidic systems, and there are a few strategies to resolve this issue. In fact, we observe the fouling of the microchannels in a bare Si/glass microfluidic device during mRNA LNP formulations (operation time (t)~10 min). As one remedy, one can implement coatings that would reduce interaction of mRNA and lipid derivatives with channel walls to prevent non-specific adsorption of materials.

Along these lines, we applied various surface coating approaches to bare Si/glass microfluidic device; i) PEG coating, ii) Zwitterion coating, iii) layer-by-layer coating, and iv) fluorine coating. Interestingly, we observe that despite applying well-known hydrophilic or hydrophobic surface coating methods, it is difficult to resolve fouling problems. Indeed, these methods typically exhibit the ability to inhibit adsorption and fouling of biomolecules in aqueous solutions. However, the practical condition for LNP formation is not simply aqueous conditions, but a mixer solution consisting of a hydrophilic aqueous solvent and an amphiphilic EtOH solvent in a 3:1 volume ratio.

Further, we note that fouling occurs due to a complex combination of various factors, including device material, solvent system, surface wettability, non-specific adsorption of biomolecules and so on.

We focus solely on preventing fouling of mRNA and LNP constituents, without the use of potentially harmful materials. Thus, we apply an omniphobic v) thin layer perfluorocarbon (TLP) coating to the Si/glass device and show that it completely repels water, EtOH, as well as water/EtOH mixture, and suppress non-specific adsorption of biomolecules such as BSA, glycine, as well as mRNA and LNP constituents. The TLP layer consists of an immobilized tethered perfluorocarbon (TP) that is overlaid by a thin liquid perfluorocarbon (LP) layer, which is FDA-approved materials. We present an antifouling resistant GMP compatible manufacturing platform, which enables high-precision (size <100 nm, encapsulation efficiency >90%) and robust production (t>50 min) of mRNA LNP. Since the TLP coating technology also prevents aggregation of nanoparticles, it opens up the possibility of creating a new class of nanomaterials and devices lined by anti-adsorption and antifouling surfaces.

### EXPERIMENTAL METHODS

### Channel Surface Coating of Microfluidic Mixing Device

The Si/glass microfluidic mixing device was flushed by infusing of 0.05M NaOH solution to activate surface of microchannels, followed by rinsing with D.I. water and EtOH.

### Lipid Nanoparticle (LNP) Formulation and Characterization

LNPs were formulated using a microfluidic mixing device or pipette mixing by rapid mixing of an aqueous phase containing RNA with an ethanol phase containing lipid components. The aqueous phase consisted of mRNA or polyA (Roche) diluted to 74μg/mL in 10 mM citrate buffer (Alpha Teknova, Inc., Hollister, CA). The ethanol phase contained D-Lin-MC3-DMA (MedChemExpress, Monmouth Junction, NJ), 1,2-distearoyl-sn-glycero-3-phosphocholine (DSPC; Avanti Polar Lipids, Alabaster, AL), cholesterol (Sigma-Aldrich, St. Louis, MO), and DMG-PEG2000 (Avanti Polar Lipids) in ethanol at molar ratios of 50%, 10%, 38.5%, and 1.5%, respectively. The ionizable lipid to mRNA weight ratio was 10:1 for all formulations.

Using the syringe pumps, the aqueous phase with RNA and the ethanol phase with lipids were infused at a flow rate ratio of 3:1 with a flow rate per mixing channel of 1.1-1.2 mL/min total volumetric flow rate. LNPs were dialyzed against 1X PBS in 20 kDa MWCO dialysis cassettes (Thermo Fisher, Waltham, MA) for four hours.

To characterize the LNPs, LNPs were analyzed by dynamic light scattering using a DynaPro Plate Reader III (Wyatt Technology, Santa Barbara, CA) to quantify hydrodynamic size, polydispersity index (PDI), and LNP concentration. RNA concentration was measured by A260 absorbance using a Tecan NanoQuant Plate (Thermo Fisher) and an Infinite M Plex plate reader (Tecan, Männedorf, Switzerland). Relative encapsulation efficiency was measured using a Ribogreen Quant-it RNA assay kit (Thermo Fisher) per the manufacturer instructions and reported as a percentage of encapsulated RNA to total RNA in the sample. For the cryo-EM imaging, 3 μL of LNPs at a concentration of ~100 ng per μL mRNA were applied to a Quantifoil holey carbon grid which had been glow discharged. Imaging was performed at the Beckman Center for cryo-EM on a Titan Krios (Thermo Scientific) equipped with a K3 Bioquantum.

### Additional Disclosure

Despite the numerous advantages demonstrated by microfluidic mixing for RNA-loaded lipid nanoparticle (RNA-LNP) production over bulk methods, such as precise size control, homogeneous distributions, higher encapsulation efficiencies, and improved reproducibility, their translation from research to commercial manufacturing remains elusive. A persistent challenge hindering the adoption of microfluidics for LNP production is the fouling of device surfaces during prolonged operation, which significantly diminishes performance and reliability. The complexity of LNP constituents, including lipids, cholesterol, RNA, and solvent mixtures, makes it difficult to find a single coating that can prevent fouling.

To address this challenge, we propose using an immobilized liquid lubricant layer, for example, perfluorodecalin (PFD), to create an antifouling surface that can repel the multiple LNP constituents. We apply this technology to an exemplary, non-limiting staggered herringbone microfluidic (SHM) mixing chip and achieve >3 hr of stable operation, a >15×increase relative to gold standard approaches. We also demonstrate the compatibility of this approach with a parallelized microfluidic platform that incorporates 256 SHM mixers, with which we demonstrate scale up, stable production at L/hr production rates suitable for commercial scale applications. We verify that the LNPs produced on our chip match both the physiochemical properties and performance for both in vitro and in vivo messenger RNA (mRNA) delivery as those made on chips without the coating. By suppressing surface fouling with an immobilized liquid lubricant layer, this technology not only enhances RNA-LNP production but also promises to transform the microfluidic manufacturing of diverse materials, ensuring more reliable and robust processes.

Lipid nanoparticles (LNPs) have emerged as highly effective delivery vehicle for mRNA vaccines and therapeutics.¹⁻³ They played a pivotal role in the Pfizer/BioNTech and Moderna COVID-19 vaccines,⁴⁻⁶ and are now revolutionizing the pharmaceutical industry as they provide unprecedented opportunities for protein replacement therapies,^{7,8} gene editing,^{9,10} and accelerated vaccine development.^{11,12}

RNA-loaded LNP (RNA-LNP) therapeutics are typically composed of an RNA, an ionizable lipid, phospholipid, cholesterol, and a polyethylene glycol-lipid conjugate (PEG-lipid); the ionizable lipid is designed to be neutrally charged at physiological pH and charged when endocytosed so it can release its RNA cargo to be delivered.^{8, 11, 13}

As the field rapidly advances, there is increasing recognition of the profound influence of the choice of the constituent materials of the LNP, as well as its size, charge, and morphology, on its function in vivo.¹³⁻¹⁶ The function of the LNP in vivo is itself also multidimensional, including its tropism to particular cell types or tissues, the immune response it elicits, its ability to induce the target cells to express the protein of interest, and its ability to traverse biological barriers such as the blood-brain barrier.^{1,14,17}

Given the pivotal role of formulation on LNP function, a critical challenge hindering the successful development of RNA-LNP drugs is the current lack of technologies that can produce a precisely defined LNP formulation across the many orders of magnitude of throughput—from discovery (mL/h) all of the way to commercial manufacturing (L/h)—all while also meeting the stringent quality standards of the pharmaceutical industry.^{7,18,19}

Microfluidics has emerged as a promising solution to this problem. Microfluidic mixing units for RNA-LNP production, which allow rapid mixing at the micrometer-scale level, have demonstrated superiority over bulk methods as they provide improved precision for controlling particle size, homogeneity of particle size distributions, morphology, encapsulation efficiencies, and reproducibility.²⁰-22 Due to the non-uniformity of mixing over the time-scales of LNP formation, the conventional bulk mixing approaches tend to result in poor encapsulation efficiency, high polydispersity and high batch-to-batch variations. The parallelization of microfluidic mixing units, where ~1 to >10⁴ units can be incorporated onto a single chip and operated in parallel, has successfully demonstrated scale-invariant, precision production of RNA-LNPs.^{18,23,24} These devices have been implemented using replica-molding of polymers, 3D printing for rapid prototyping and silicon and glass (Si/glass). The Si/glass platform is particularly well suited for pharmaceutical manufacturing because of its solvent compatibility and the ability to operate at high pressures.^{18,25-27}

However, despite the advantages offered by microfluidic mixing units in LNP production, a significant challenge arises from the fouling of RNA-LNP constituents on the surface of the microfluidic device during operation. Over time, such a phenomenon significantly degrades the performance of the device through the loss of particle homogeneity and a significant reduction in encapsulation efficiency; moreover, fouling ultimately induces the catastrophic failure of the device due to clogging.²⁷⁻²⁹ The inability to effectively prevent fouling in these devices likely impedes the widespread adoption of continuous manufacturing using microfluidic technologies.^{19,30,31}

Previous approaches to prevent fouling in microfluidic devices employ surface modifications such as polyethylene glycol (PEG) coating,^{32,33} zwitterionic coating,^{34,35} layer-by-layer (LbL) coating,^{28,36} and amine-/hydroxy-/fluorine-based coating.^{37,38} However, these methods were primarily designed to prevent biomolecule adsorption in aqueous environments and have not been successfully applied to the RNA LNP manufacturing process, which involves solvent mixtures of water/ethanol for precipitation and self-assembly.

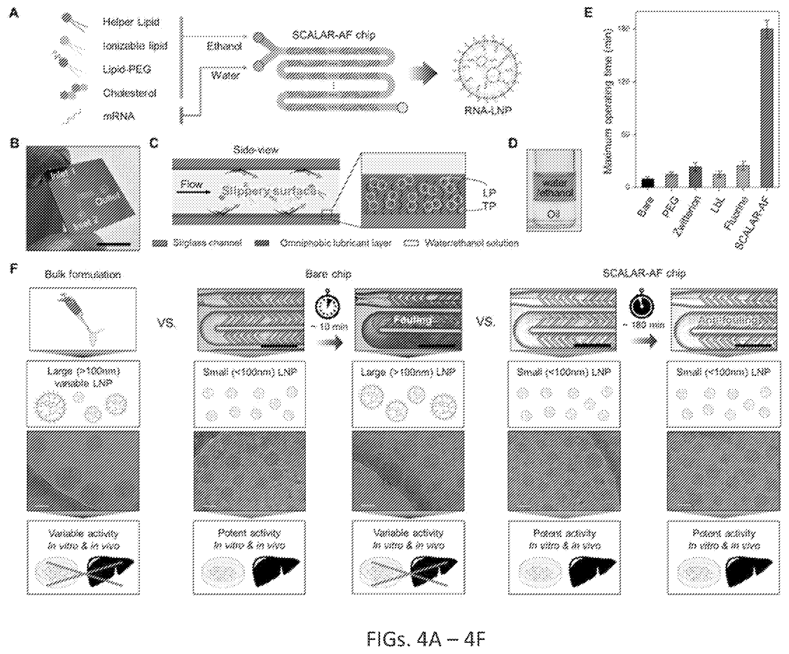

Here, we address this challenge by developing a microfluidic chip that incorporates a lubricant oil layer coating to inhibit fouling, enabling continuous and robust manufacturing of RNA-LNPs (FIG. 4). Our microfluidic mixing chip, termed SCALAR-AF (Silicon Scalable Lipid Nanoparticle Generation-Anti Fouling platform), is entirely fabricated with silicon and glass (Si/glass) substrates to meet the manufacturing requirements of the pharmaceutical industry (FIG. 4B).²⁵

When the Si/glass chip's channels are uncoated, fouling occurs within 10 min into the production of RNA-LNPs, a phenomenon also observed with polydimethylsiloxane (PDMS) and other polymer microfluidic chips (FIG. 4E). We evaluate various gold standard strategies to prevent fouling, including coating with PEG, zwitterion, layer-by-layer (LbL), and fluorine coatings; however, fouling occurred within 20 min for all coatings.

To address this challenge, we develop an alternative approach wherein we protect the surface from fouling using a tethered oil lubrication coating of liquid perfluorocarbon (TLP) immobilized to the surface of the microfluidic channels using a perfluorinated surface (FIG. 4C, D).37,39-41 The TLP surface coating, which is immiscible with the mixture of water and ethanol, forms a slippery surface that suppresses fouling of the microfluidic channels during RNA-LNP formation. As an illustration, we employ PFD oil, a lubricant oil approved by the U.S. Food and Drug Administration (FDA) for various therapeutic and biomedical applications, such as blood substitutes, cancer therapy, and diagnosis.^{37,39,42} The SCALAR-AF approach successfully demonstrates continuous and robust production of RNA-LNPs for at least 3 hr (>15-fold increase compared to the bare chip) without fouling (FIG. 4E, F). To illustrate its applicability to scaled-up production, we design a microfluidic chip that integrates 256 parallel mixing units on a single 4-inch chip, with every generator's surface coated with TLP to prevent fouling; this chip can generate RNA-LNPs at L/hr throughputs.

We characterize the performance of our SCALAR-AF chip using a gold standard formulation previously optimized for siRNA and mRNA delivery, demonstrating robust LNP production. LNPs produced using SCALAR-AF chips are validated to have physiochemical properties and performance in both in vitro and in vivo model systems that matched those of uncoated chips at early time points before fouling. Beyond the LNPs generated in this manuscript, we envision this technology finding broad utility as an antifouling lubricant layer coating in microfluidic chips for applications requiring continuous and robust production of functional particles used in drug delivery systems, cosmetic products, optical displays, pharmaceuticals, and foodstuffs.

### RESULTS AND DISCUSSION

### Evaluation of Conventional Surface Coatings to Prevent Fouling in the Microfluidic Generation of RNA-LNPs

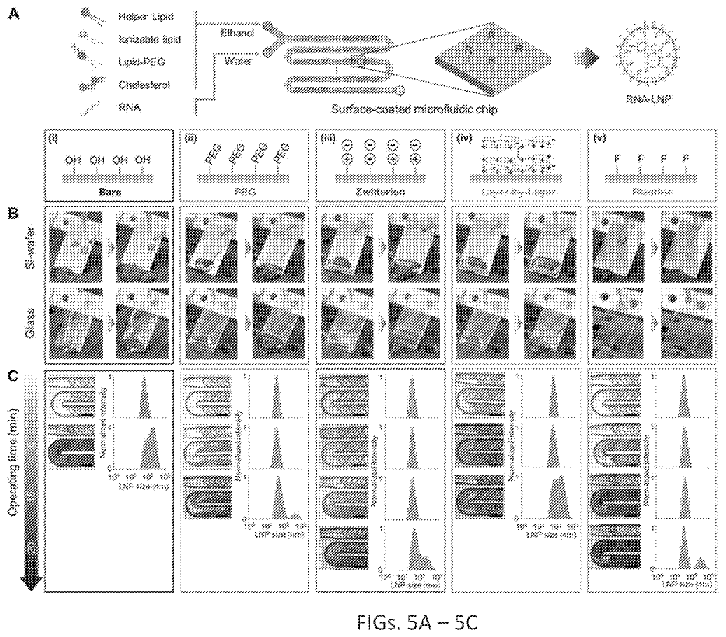

We first evaluate the anti-fouling performance of various coatings that have shown anti-fouling properties in the literature. These coatings, some of which are hydrophobic and some of which are hydrophilic, include PEG coating (PEG-silane),⁴³ zwitterion coating (sulfobetaine-silane),^{44,45} LbL coating (poly(acrylic acid)/polyacrylamide multilayers)³⁶ and fluorine coating (perfluoro-silane)(FIG. 5A).^{37,39-}41 We confirm the modification of Si and glass surfaces with these coatings by quantifying their water contact angle in air. Upon surface modification, the water contact angles change dramatically from 37°±3 for Si wafer and 47°±4 for glass substrates to 14°±3, 9°±3, and 12°±2 upon PEG, zwitterion, and LbL coatings, respectively; these values are consistent with those reported in prior publications.²⁷⁻³³ In contrast, the fluorine coating renders the surface hydrophobic with a contact angle value of 109°±5. When a mixture of water and ethanol in a 3:1 volume ratio, identical to the solvent condition for RNA-LNP formulation, is placed on these surfaces that are tilted at 45°, the liquid spreads extensively and stays on the three hydrophilic surfaces (FIG. 5B), indicating strong interactions between the mixture and the modified surfaces. In contrast, the perfluorosilane-treated substrate does not allow for extensive spreading of such a mixture, often resulting in pinned droplets on the tilted substrate (FIG. 5B).

To test the anti-fouling performance of these coatings in a microfluidic mixing device used to generate RNA-LNP, we prepare a single microfluidic mixing chip on a silicon and glass (Si/glass) substrates (details of these coatings on the surface of the SHM via a flow technique are as described in Experimental methods). We employ a staggered herringbone micromixer (SHM) design, characterized by short mixing times of less than 10 ms, resulting in highly uniform and small lipid nanoparticles (LNPs) (PDI<0.2 and average size <60 nm).^{27, 46} To evaluate our system we use an LNP formulation that includes DLin-MC3-DMA (MC3) with phospholipid distearoylphosphatidylcholine (DSPC), cholesterol, and PEG-lipid (DMG-PEG 2000) at a molar ratio of 50:10:38.5:1.5, respectively; this composition is similar to those found in the Comirnaty and Spikevax COVID-19 mRNA vaccines developed by Pfizer/BioNTech and Moderna, respectively (FIG. 5A).⁴⁻⁶

PolyA is selected as a surrogate for RNA to evaluate the durability and performance of surface-modified devices, owing to the high costs associated with using functional RNA. We infuse an aqueous polyA solution and an ethanolic lipid solution into each surface-modified chip with a flow rate ratio of 3:1 at a total flow rate of 1.2 mL/min by using syringe pumps or pressure driven pumps.

Within 10 min of the infusion of the two solutions, we observe severe fouling in the bare Si/glass chip, (FIG. 5C). Dynamic light scattering (DLS) of the product stream shows that the particle size increases significantly from 70 nm to 200 nm in this short time span, and relatedly the uniformity of the RNA-LNPs is significantly decreased, with the polydispersity index (PDI) increasing from 0.1 to 0.5 (FIG. 5C).

We next evaluate the four surface coatings described above (PEG, zwitterionic, LbL, and perfluorinated silane) to prevent fouling in the single microfluidic staggered herringbone chip. Although the surface fouling proceeds more slowly compared to the bare device, all of these coatings fail within 20 min. For all coatings, fouling become apparent in microscopy within 15 min of operating the device and led to channel blockages within 20 to 25 min (FIG. 5C). Among the four coatings, zwitterionic surface treatment provides the longest antifouling behavior; yet, the device fails in less than 20 min.

As fouling progresses, the size of the produced LNPs become larger, exceeding 150 nm, and their size distribution becoming broader, often exhibiting a bimodal pattern. Although various hydrophilic and hydrophobic coatings are widely studied for their ability to prevent fouling, they are known for inhibiting adsorption and fouling of biomolecules in fully aqueous solutions.⁴²

In contrast, LNP formation involves a mixture of water and ethanol and the precipitation and self-assembly of four lipids and RNA, leading to a highly complex microenvironment that promotes the deposition of materials onto solid surfaces. Thus, commonly reported hydrophilic or hydrophobic coatings that are designed to keep water molecules tightly bound to or away from the surface are not suitable for resolving the fouling issue that arises during the LNP manufacturing process.

### Continuous and Robust Manufacturing of RNA-LNPs from SCALAR-AF Chip

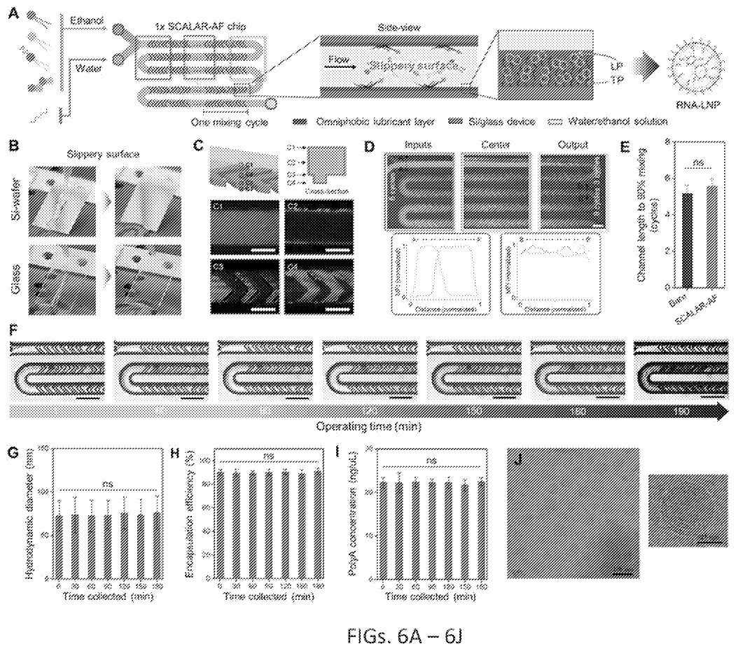

We next evaluate the TLP coating that TP is overlaid by a thin LP layer to prevent fouling on our chip (FIG. 6A). We confirm the modification of Si and glass surfaces with TLP coatings by quantifying their water contact angle in air. The water contact angles on TLP-coated planar Si and glass substrates are 1200±5 and 119°±6, respectively; these values are higher compared to those observed for surfaces coated with a simple perfluorosilane and are consistent with literature.^{37,39-41} Moreover, we observe that the TLP-coated substrates provide excellent slipperiness against the water/ethanol mixture, effectively preventing the fluid retention or adhesion on the substrates (FIG. 6B). To coat our chips with TLP, we (1) create reactive-OH groups on the surface by exposing the surface of the microfluidic channels and SHMs to a NaOH solution; (2) form a covalently bonded perfluorinated surface by flowing the tethered perfluorocabon (TP) silane solution through the chip, followed by heating in an oven at 60 ° C. for overnight; (3) apply the liquid perfluorocarbon (LP) oil layer by flowing the PFD into the chip, and (4) remove the excess PFD with water phase by using a syringe pump (details of TLP coating on the surface of the SHM via a flow technique are as described in Experimental methods). We denote the TLP-coated microfluidic single chip as 1×SCALAR-AF chip.

Confocal microscopy of the Rhodamine-labeled (red fluorescent dye) lubricant layer on the three dimensionally structured microfluidic channels shows conformal coating on the channel floor, ceiling, walls and features of the SHM patterns (FIG. 6C).

The mixing efficiency of the 1×SCALAR-AF chip is tested by monitoring the mixing of the aqueous and ethanolic solutions by adding fluorescein isothiocyanate (FITC) dextran and Nile red in the two solutions, respectively (FIG. 6D).

These two dyes are selected since they approximate the size of the lipids and RNA, respectively.²⁵ The mixing efficiency is evaluated by quantifying the channel length at which the two dyes are 90% mixed (FIG. 6E).

At a flow rate ratio of 0.3, which is the lipid to nucleic acid ratio, the 1×SCALAR-AF chip allows excellent mixing efficiency, consistent with prior results obtained with a pristine Si/glass device.⁴⁶ The 1x SCALAR-AF chip continuously produce LNPs for at least ~180 min without severe fouling, a marked improvement over the other anti-fouling coatings tested which all failed within 20 min (FIG. 6F). The physical characteristics of polyA LNPs show no significant changes over this time period, as evidenced consistent hydrodynamic diameter, encapsulation efficiency, and RNA concentration throughout the 3-h period (FIG. 6G-I). In addition, the cryogenic TEM images of the resulting poly A LNPs demonstrate the high uniformity of LNPs in size and morphology (FIG. 6J). We notice that the 1×SCALAR-AF chip start showing evidence of surface fouling after 3 hr likely due to the depletion of the lubricant oil under shear as well as its diffusion into the flowing water/ethanol mixture stream.

### Scaling-Up the Production of RNA LNP via Parallelization and Surface Modification

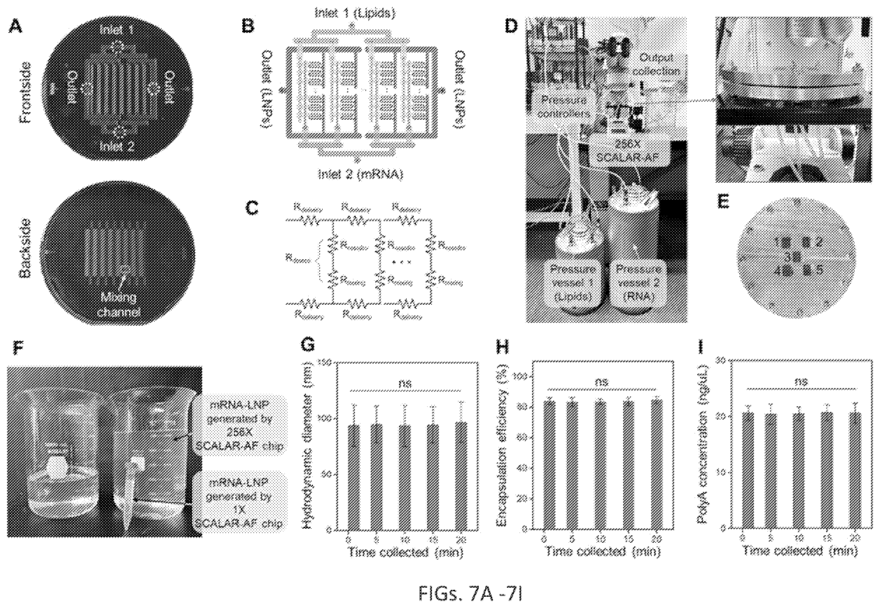

To demonstrate the potential of this approach for industrial scale manufacturing of RNA-LNPs, we fabricate a SCALAR-AF microfluidic chip that parallelizes 256 microfluidic SHM devices (FIG. 7A). We use a ladder design for parallelization to distribute fluids to and collect fluids from individual mixing units (FIG. 7B).^{47,48} The effective resistance of a single mixing device (R_{device}), which is the sum of the resistance of a flow resistor (Rᵣₑₛᵢₛₜₒᵣ) and that of the mixer (R_{mixing}), is kept to be significantly higher than the resistance of the delivery channel between two devices (FIG. 7C), satisfying the previously reported design rule: 2N (R_{device}/R_{delivery})<0.01.⁴⁸ The TLP coating approach can be readily extended to the parallelized 256×SCALAR-AF chip thanks to the simplicity of the method as well as the uniform flow distribution that the chip design ensures. In addition, we observe no considerable alteration in the flow distribution and mixing efficiency of the parallelized 256×SCALAR-AF chip.

To validate that the 256×SCALAR-AF chip can formulate LNPs at a large scale without compromising the physical characteristics of LNPs, we produce RNA-LNPs and compare their physical properties such as hydrodynamic diameter, RNA concentration, and encapsulation efficiency with those generated using a 1×SCALAR-AF. We use C12-200, another gold-standard ionizable lipid with DSPC, cholesterol, and DMG-PEG. We use polyA as our RNA and C12-200 as the ionizable lipid, to make this high throughput experiment financially feasible.

We inject the poly A aqueous and ethanolic lipid solutions into each input port of the 256×SCALAR-AF chip with a flow rate ratio of 3:1 at a total flow rate of 14.4 L/hr using our custom-built pressure-driven flow system (FIG. 7D). The chip is able to produce poly A LNPs for 20 min without fouling with a resultant volume of 4.8 L, which is equivalent to 2.4 g of RNA or 9,600 vaccines doses (FIG. 7F). Additionally, we confirm the LNP hydrodynamic diameter (~80 nm), encapsulation efficiency (>80%), and the RNA concentration (~22.5 ng/μL) measured during the 20 min operation of the parallelized chip are comparable to those measured for resulting formulation from a single SCALAR-AF chip (FIG. 7G-I).

### In Vitro and in Vivo Validation of RNA LNPs Prepared From Scalar-AF Chip

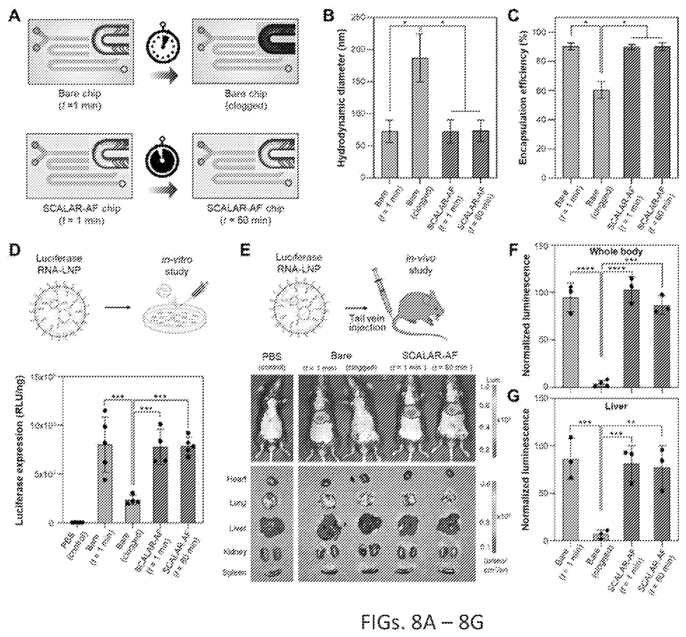

We evaluate the potential impact of TLP-coating on the potency of RNA LNPs. To this end, we use the TLP-coated SCALAR-AF to formulate luciferase RNA-LNPs and compare their physical properties as well as their in vitro and in vivo activity to LNPs generated by a bare chip (t=1 min and after clogging) (FIG. 8A). We encapsulate RNA encoding luciferase in LNPs composed of MC3, a well-validated ionizable lipid that has been FDA approved for RNA delivery, phospholipid DSPC, cholesterol, and lipid-anchored PEG (DMG-PEG 2000) at a molar ratio of 50:10:38.5:1.5.

While the LNPs generated by bare chips after severe fouling have a dramatic increase in their size (>150 nm) and size heterogeneity (PDI>0.4); in contrast, LNPs generated by the 1×SCALAR-AF chip at t=1 min and 60 min show no significant changes in the hydrodynamic size (<80 nm) and size distribution (PDI<0.2) (FIG. 8B). In addition, encapsulation efficiency measurements, quantified by a RiboGreen assay, demonstrate that 1×SCALAR-AF chip maintains a high encapsulation efficiency (>85%) after 60 min, whereas the encapsulation efficiency of the bare chip degrade significantly to ~60% over the same period of time (FIG. 8C).

For in vitro evaluation, Hela cells are selected to demonstrate a potential application for cell engineering (i.e., gene delivery) using RNA-LNPs (FIG. 8D). We deliver luciferase-encoding RNA-LNPs to Hela cells at doses of 60 ng RNA per 60k cells. We observe that while the luciferase expression of luciferase RNA-LNPs formulated by 1×SCALAR-AF chip at t=1 min and 60 min show high activity, the potency of LNPs produced by a bare chip after severe fouling degrades by 4-fold compared to LNPs produced at t=1 min (FIG. 8D). For in vivo testing, these RNA-LNPs are administered to C57BL/6 mice via tail vein injection at 4 μg per mouse, and we quantify the luminescent signal in the whole body and dissected major organs (i.e., heart, lung, liver, spleen, and kidneys) after 6 hr (FIG. 8E). We observe that there are no significant differences between the whole-body and liver luminescent signals for RNA-LNPs prepared using the 1×SCALAR-AF chip at both t=1 min and 60 min (FIG. 8F, G). Although RNA-LNPs produced from the bare chip at t=1 min show a strong luminescent signal, the potency of the LNPs degrades dramatically to the point where little luminescence is detected in the whole-body and in the liver on the device is fouled and clogged (FIG. 8F, G). The poor performance of the LNPs produced from bare chips after fouling is likely due to the larger LNP size (>150 nm) as well as lower encapsulation efficiency (~60%) leading to rapid blood clearance by the reticuloendothelial system and limited passage through liver fenestrations.^{11,15,49} Overall, these results demonstrate that our anti-fouling TLP chip can formulate highly potent RNA-LNPs for potential applications of RNA therapeutics and vaccines.

### Conclusions

We present a solution to the persistent challenge of fouling in microfluidic mixing devices for RNA-LNPs production. We have successfully created an antifouling surface capable of repelling the diverse constituents of LNPs in the mixture of solvents used in their production. This new approach, applied to a SHM mixing chip, extends stable operation to over 3 hr, representing a substantial improvement over conventional methods. Furthermore, our approach is scalable, as demonstrated by its compatibility with a parallelized microfluidic platform incorporating 256 SHM mixers, achieving production rates suitable for commercial applications. Importantly, we confirm that LNPs produced using our coated chip maintain identical physicochemical properties and performance for both in vitro and in vivo RNA delivery, compared to uncoated devices at short time-scales before they foul, validating the efficacy of our antifouling strategy in maintaining LNP quality.

### Experimental Methods

Chip Fabrication. In the case of a single 1×chip, the process involves removing the processes of layer 1, with all other procedures remaining identical. The chips were fabricated in the Quattrone Nanofabrication Facility at the University of Pennsylvania. The chip has four mask layers which were designed in AutoCAD (Autodesk, San Rafael, CA) to define the delivery channels (layer 1), mixing channels (layer 2), herringbones and resistors (layer 3), and through silicon vias (etching layer 4;layer 5 overall). The design of each layer was patterned onto chrome-coated soda lime photomasks (AZ1500; Telic Company, Santa Clarita, CA) using a Heidelberg laser writer with a 10 mm write head. Photomasks were developed in AZ 300MIF developer (EMD Performance Materials Corp., Philadelphia, PA) in chromium etchant (Transene Company, Danvers, MA); the remaining photoresist was removed by Remover 1165 (DuPont, Wilmington, DE). To fabricate the chip, each layer was lithographically patterned onto a single 100 mm silicon wafer (ID 775; University Wafer, South Boston, MA) followed by deep reactive ion etching (DRIE) to define the channels. For the lithography, S1805 photoresist (Dow, Midland, MI) the resist was spray coated to the desired thickness using an AS8 Alta Spray Coater (SUSS MicroTec, Garching, Germany).

In the first etching layer, the wafer was dipped in 49% hydrofluoric acid (HF), then 16 μm of photoresist was spray-coated onto the front side of the silicon wafer. Then, the wafer was soft baked at 110° C. for 10 min. The delivery channel design was exposed using a MA6 mask aligner (SUSS MicroTec), then the wafer was baked at 110° C. for 5 min. The wafer was developed in AZ 300MIF developer, rinsed with water, dried with N₂, and baked at 110° C. for 10 min. The wafer was etched using DRIE (SPTS Rapier Si DRIE, Newport, UK) to a 370 μm etch depth, cleaned using acetone and isopropanol (IPA) for 5 min each, then cleaned by immersion in Nano-Strip (CMC Materials, Aurora, IL), followed by cleaning in a Spin Rinse Dryer (SRD; RENA Compass, RENA, Albany, OR). For the second etching layer, the wafer was dipped in 49% HF, then the backside of the wafer was coated with 8 μm of photoresist, then baked at 110° C. for 5 min, then exposed with the mixing channel design. Then, the wafer was developed and baked at 110° C. for 5 min, then etched to a 70 μm etch depth using DRIE. The wafer was then cleaned using acetone, IPA, Nano-Strip, and SRD as before. For the third etching layer, the wafer was dipped in 49% HF, then the backside of the wafer was coated with 4 μm of photoresist and baked in an oven at 110° C. for 5 min, then developed and baked in an oven at 110° C. for 5 min, etched to a 25 μm etch depth using DRIE. Using plasma-enhanced chemical vapor deposition (Oxford Plasma Lab 100 PECVD) (Oxford Instruments, Abingdon, UK), 4 μm of SiO₂ was deposited, then the wafer was cleaned using Nano-Strip and SRD. For the fourth etching layer, the frontside of the wafer was coated with 8 μm of photoresist and baked at 110 ° C. for 5 min, then exposed with the through silicon via design. The wafer was developed and baked at 110° C. for 5 min, then etched to a 60 μm etch depth using DRIE. The wafer was then cleaned using acetone and IPA as before, then in 25% HF for 5 min. The etched silicon wafer and a 100 mm Borofloat 33 glass wafer (ID 517; University Wafer) were cleaned using Nano-Strip and SRD as before. Another Borofloat 33 glass wafer was micromachined with 1 mm holes that serve as inlets and outlets, then this wafer was cleaned using Nano-Strip and SRD as before. Finally, for 1×chip, a glass wafer was anodically bonded on one side of Si wafer. For 256×chip, the two glass wafers and one Si wafer were stacked together and anodically bonded on each side using an EVG 510 Wafer Bonding System (EVG Group, Oberosterreich, Austria) at 900 Volts with a 1000 N for 1 hr.

Surface Coating Methods. Basically, to coat our chips with TLP, the Si/glass microfluidic mixing chip was flushed by infusing of 0.01M NaOH solution to activate the hydroxyl group (—OH) on the surface of Si/glass microchannels for 30 min at room temperature, followed by rinsing with water and ethanol. Then, we formed a covalently bonded tethered perfluorocarbon (TP) surface by flowing the liquid silane solution (5%v/v trichloro(1H,1H,2H,2H-perfluorooctyl)silane (Sigma Aldrich) in anhydrous ethanol (Sigma Aldrich)) for 6 hr at room temperature. The chip was rinsed with anhydrous ethanol and gently heated in an oven with desiccant at 60° C. for overnight. We applied the liquid perfluorocarbon (LP) oil layer into the channel by flowing the perfluorodecalin (PFD, Sigma Aldrich) oil, and followed removing excess PFD with water phase by using a syringe pump at the flow rate of 1 mL/h.

For different coatings, some of which are hydrophobic and some of which are hydrophilic, include PEG coating (PEG-silane),⁴³ zwitterion coating (sulfobetaine-silane),^{44, 45} LbL coating (poly(acrylic acid)/polyacrylamide multilayers)³⁶ and fluorine coating (perfluoro-silane)^{37, 39-41} These coating methods were prepared based on prior papers, followed by flow surface coating methods.

For PEG coating, we formed a covalently bonded PEG surface by flowing the liquid silane solution (15 w % mPEG-Silane (Mn 5 000, Sigma Aldrich) in dimethyl sulfoxide (DMSO, Sigma Aldrich)) for 1 hr at 90° C., followed by rinsing with DMSO.

For zwitterion coating, we formed a covalently bonded zwitterion surface by flowing a liquid sulfobetaine silane solution (10 mM SBSi (3-(dimethyl(3-trimethoxysilyl)propyl)ammonia propane-1-sulfonate, Gelest) in pH 3.5 DI water) for 6 hr at room temperature, followed by rinsing with pH 3 DI water.

For LbL coating, we formed the layer-by-layer (LbL) assembly of multilayer films with oppositely charged polymers on surfaces by alternately flowing a liquid 10 mM solutions of poly(acrylic acid) (PAA, M_{w}~50 000, 25% aqueous solution, Sigma Aldrich) and polyacrylamide (PAAm, M_{w}~5 000 000-6 000 000, Sigma Aldrich) in pH 3 DI water. For 10 bilayers of a PAA/PAAm film inside microfluidic chip, PAA solution and the PAAm solution (for 10 min each) with a rinse step in pH 3 DI water for 2 min between the two polymer adsorption steps. After LbL assembly, the PAA/PAAm is treated in a vacuum oven for 8 hr at 180° C.

For fluorine coating, we formed a covalently bonded fluorine surface by flowing the liquid silane solution (5% v/v trichloro(1H, 1H,2H,2H-perfluorooctyl)silane (Sigma Aldrich) in anhydrous ethanol (Sigma Aldrich)) for 6 hr at room temperature, followed by rinsing with anhydrous ethanol.

Lipid Synthesis. The ionizable lipid C12-200 was synthesized by adding N1-(2-(4-(2-aminoethyl)piperazin-1-yl)ethyl)ethane-1,2-diamine (1.00 g, 4.64 mmol, 1.0 equiv.), ethanol (10 mL), and 1,2-epoxydodecane (7.10 mL, 32.5 mmol, 7.0 equiv.) to a 50 mL round bottom flask with a stir bar. The reaction was stirred at 80° C. for 48 hr.

Afterwards, the ethanol was removed in vacuo and then diluted in 15 mL dichloromethane (DCM). The compound was purified using a CombiFlash (Teledyne ISCO, Lincoln, NE) using a 40 g RediSep Gold® silica gel flash column using a gradient mobile phase from 100% DCM to 20% DCM and 80% Ultra solution over 30 min. The solution was run purified in two runs, using half of the DCM mixture each time. C12-200 was isolated as a yellow-orange oil in 37% yield and characterized by ¹H NMR. ¹H NMR spectra were obtained on a NEO 400 MHz spectrometer using deuterated chloroform as the solvent. ¹H NMR (400 MHZ, CDl₃) δ 4.99-3.30 (m, 8H), 3.14-1.99 (m, 30H), 1.84-1.05 (m, 90H), 1.05-0.67 (m, 15H).

RNA-LNPs Formulation using 1× and 256×SCALAR-AF Chips. RNA-LNPs were formulated using a microfluidic mixing device or pipette mixing by rapid mixing of an aqueous phase containing RNA with an ethanol phase containing lipid components. The aqueous phase consisted of luciferase RNA (TriLink) or polyA (Roche) diluted to 24.7μg/mL in 10 mM citrate buffer (Alpha Teknova, Inc., Hollister, CA). The ethanol phase contained D-Lin-MC3-DMA (MedChemExpress, Monmouth Junction, NJ), 1,2-distearoyl-sn-glycero-3-phosphocholine (DSPC; Avanti Polar Lipids, Alabaster, AL), cholesterol (Sigma-Aldrich, St. Louis, MO), and DMG-PEG 2000 (Avanti Polar Lipids) in ethanol at molar ratios of 50%, 10%, 38.5%, and 1.5%, respectively. The ionizable lipid to RNA weight ratio was 10:1 for all formulations. Using the syringe pumps or the custom pressure-driven flow system, the aqueous phase with RNA and the ethanol phase with lipids were infused at a flow rate ratio of 3:1 with a total volumetric flow rate of 1.2 mL/min.

For the large scale RNA-LNPs formulation, RNA-LNPs were formulated with the synthesized ionizable lipid C12-200 instead of MC3. The aqueous phase consisted of poly A diluted to 24.7 μg/mL in 10 mM citrate buffer (Alpha Teknova, Inc., Hollister, CA). The ethanol phase contained C12-200, 1,2-distearoyl-sn-glycero-3-phosphocholine (DSPC), cholesterol, and DMG-PEG 2000 in ethanol at molar ratios of 50%, 10%, 38.5%, and 1.5%, respectively. The ionizable lipid to RNA weight ratio was 10:1 for all formulations. A custom-built pressure driven flow system (FIG. 7D) rated to 100 pounds per square inch (psi) delivered fluids to the chip. A nitrogen tank was connected to two dual-valve pressure controllers (Alicat Scientific, Tucson, AZ) that controlled the RNA and lipid inputs using Flow Vision 2.0 software (Alicat Scientific).

The pressure controllers were connected to 5-gallon and 3-gallon T-304 stainless steel pressure vessels (Alloy Products Corp., Waukesha, WI), where the 5-gallon vessel was used for the RNA input and the 3-gallon vessel was used for the lipids input. Connections between the nitrogen tank, pressure controllers, and pressure vessels used PTFE tubing with a ¼″ OD (Part 52365K 61; McMaster-Carr, Elmhurst, IL). Pressure vessels were connected to the chip using ¼″ OD PTFE tubing and ⅛″ OD FEP tubing (Part 52355K41; McMaster-Carr). The housing system was mounted on an xyz translational stage to monitor individual device performance during operation. LNPs were dialyzed against 1×PBS in 20 kDa MWCO dialysis cassettes (Thermo Fisher, Waltham, MA) for 4 hr.

Characterization of RNA-LNPs. To characterize the LNPs, dynamic light scattering (DLS) was performed using a DynaPro Plate Reader III (Wyatt Technology, Santa Barbara, CA) to quantify hydrodynamic diameter and polydispersity index (PDI). Sizes reported are intensity weighted averages, and standard deviation of LNP size is calculated by: σ=(PDI*diameter²)^{1/2}. RNA concentration was measured by A260 absorbance using a Tecan NanoQuant Plate (Thermo Fisher) and an Infinite M Plex plate reader (Tecan, Männedorf, Switzerland). Relative encapsulation efficiency was measured using a Ribogreen Quant-it RNA assay kit (Thermo Fisher) per the manufacturer instructions and reported as a percentage of encapsulated RNA to total RNA in the sample. For the cryo-EM imaging, 3 μL of LNPs at a concentration of ~100 ng per AL RNA were applied to a Quantifoil holey carbon grid which had been glow discharged. Grids were blotted and plunge frozen in liquid ethane using a Vitrobot Mark IV (Thermo Scientific). Imaging was performed at the Beckman Center for cryo-EM on a Titan Krios (Thermo Scientific) equipped with a K3 Bioquantum.

RNA Delivery Studies in vitro. Hela cells, modified to express firefly luciferase, were cultured in Dulbecco's Modified Eagles Media (DMEM) (Thermo Fisher) supplemented with 10% fetal bovine serum and 1% penicillin/streptomycin. The cells were incubated at 37° C. in 5% CO₂. Hela cells were counted and combined at a 1:1 ratio, plated at 10k cells per well in a 96-well plate (Thermo Fisher) and treated with luciferase RNA-LNPs; luminescence was measured 6 hr later using a Luciferase Assay System (Promega) according to the manufacturer's protocol. The luminescent signal was normalized to untreated 5 cells after subtracting the background (wells without cells with reagents). To assess cytotoxicity, a Cell Titer-Glo Luminescent Cell Viability Assay (Promega) was performed according to manufacturer's instructions, and luminescence was normalized to untreated cells after subtracting the background.

Animal Studies. All animal procedures were performed on female C 56BL/6 mice aged 6-8 weeks (The Jackson Laboratory) in accordance with protocols approved by the Institutional Animal Care and Use Committee (IACUC) of the University of Pennsylvania. For the luciferase study, mice were injected by intramuscular injection with luciferase RNA-LNPs at an RNA dose of 4 μg per mouse. At 6 hr post injection, mice were intraperitoneally injected with D-luciferin potassium salt at 150 mg/kg.

Bioluminescence imaging of the whole body and dissected major organs was performed on an in vivo imaging system (IVIS; PerkinElmer, Waltham, MA). Bioluminescence values were quantified by measuring photon flux in the region of interest using Living Image 4.7.3 software (PerkinElmer) and photon flux values were normalized to the background of each image.

Statistical Information. Statistical tests were performed by GraphPad Prism 9. Data are presented as mean ±standard deviation unless otherwise specified. One-way analysis of variance (ANOVA) and Tukey's multiple comparisons test were used to compare between two groups or several groups, respectively. p<0.05 was considered statistically significant.

### REFERENCES

(1) Pardi, N.; Hogan, M. J.; Porter, F. W.; Weissman, D. mRNA vaccines—a new era in vaccinology. Nat. Rev. Drug. Discov. 2018, 17 (4), 261-279.
(2) Pardi, N.; Hogan, M. J.; Weissman, D. Recent advances in mRNA vaccine technology. Curr. Opin. Immunol. 2020, 65, 14-20.
(3) Reichmuth, A. M.; Oberli, M. A.; Jaklenec, A.; Langer, R.; Blankschtein, D. mRNA vaccine delivery using lipid nanoparticles. Ther. Deliv. 2016, 7 (5), 319-334.
(4) Milane, L.; Amiji, M. Clinical approval of nanotechnology-based SARS-COV-2 mRNA vaccines: impact on translational nanomedicine. Drug Deliv. Transl. Res. 2021, 11 (4), 1309-1315.
(5) Polack, F. P.; Thomas, S. J.; Kitchin, N.; Absalon, J.; Gurtman, A.; Lockhart, S.; Perez, J. L.; Pérez Marc, G.; Moreira, E. D.; Zerbini, C.; et al. Safety and Efficacy of the BNT16b2b2 mRNA Covid-19 Vaccine. N. Engl. J. Med. 2020, 383(27), 2603-2615.
(6) Baden, L. R.; El Sahly, H. M.; Essink, B.; Kotloff, K.; Frey, S.; Novak, R.; Diemert, D. Spector, S. A.; Rouphael, N.; Creech, C. B.; et al. Efficacy and Safety of the mRNA-1273 SARS-COV-2 Vaccine. N. Engl. J. Med. 2021, 384 (5), 403-416.
(7) Semple, S. C.; Leone, R.; Barbosa, C. J.; Tam, Y. K.; Lin, P. J. C. Lipid Nanoparticle Delivery Systems to Enable mRNA-Based Therapeutics. Pharmaceutics 2022, 14 (2), 398.
(8) Mehta, M.; Bui, T. A.; Yang, X.; Aksoy, Y.; Goldys, E. M.; Deng, W. Lipid-Based Nanoparticles for Drug/Gene Delivery: An Overview of the Production Techniques and Difficulties Encountered in Their Industrial Development. ACS Mater. Au 2023, 3 (6), 600-619.
(9) Guevara, M. L.; Persano, F.; Persano, S. Advances in Lipid Nanoparticles for mRNA-Based Cancer Immunotherapy. Front. Chem. 2020, 8, Review.
(10) Kulkarni, J. A.; Cullis, P. R.; van der Meel, R. Lipid Nanoparticles Enabling Gene Therapies: From Concepts to Clinical Utility. Nucleic Acid Ther. 2018, 28 (3), 146-157.
(11) Hou, X.; Zaks, T.; Langer, R.; Dong, Y. Lipid nanoparticles for mRNA delivery. Nat. Rev. Mater. 2021, 6(12 ), 1078-1094.
(12) Mulligan, M. J.; Lyke, K. E.; Kitchin, N.; Absalon, J.; Gurtman, A.; Lockhart, S.; Neuzil, K.; Raabe, V.; Bailey, R.; Swanson, K. A.; et al. Phase I/II study of COVID-19 RNA vaccine BNT162b1 in adults. Nature 2020, 586 (7830), 589-593.
(13) Kowalski, P. S.; Rudra, A.; Miao, L., Anderson, D. G. Delivering the Messenger: Advances in Technologies for Therapeutic mRNA Delivery. Mol. Ther. 2019, 27 (4), 710-728.
(14) Jung, H. N.; Lee, S. Y.; Lee, S.; Youn, H.; Im, H. J. Lipid nanoparticles for delivery of RNA therapeutics: Current status and the role of in vivo imaging. Theranostics 2022, 12 (17), 7509-7531.
(15) Hassett, K. J.; Benenato, K. E.; Jacquinet, E.; Lee, A.; Woods, A.; Yuzhakov, O.; Himansu, S.; Deterling, J.; Geilich, B. M.; Ketova, T.; et al. Optimization of Lipid Nanoparticles for Intramuscular Administration of mRNA Vaccines. Mol. Ther. Nucleic Acids 2019, 15, 1-11.
(16) Nakamura, T.; Kawai, M.; Sato, Y.; Maeki, M.; Tokeshi, M.; Harashima, H. The Effect of Size and Charge of Lipid Nanoparticles Prepared by Microfluidic Mixing on Their Lymph Node Transitivity and Distribution. Mol. Pharmaceutics 2020, 17 (3), 944-953.
(17) Hashiba, A.; Toyooka, M.; Sato, Y.; Maeki, M.; Tokeshi, M.; Harashima, H. The use of design of experiments with multiple responses to determine optimal formulations for in vivo hepatic mRNA delivery. J. Control. Release 2020, 327, 467-476.
(18) Shepherd, S. J.; Warzecha, C. C.; Yadavali, S.; El-Mayta, R.; Alameh, M.-G.; Wang, L.; Weissman, D.; Wilson, J. M. Issadore, D.; Mitchell, M. J. Scalable mRNA and siRNA Lipid Nanoparticle Production Using a Parallelized Microfluidic Device. Nano Lett. 2021, 21 (13), 5671-5680.
(19) Webb, C.; Forbes, N.; Roces, C. B.; Anderluzzi, G.; Lou, G.; Abraham, S.; Ingalls, L.; Marshall, K.; Leaver, T. J.; Watts, J. A.; et al. Using microfluidics for scalable manufacturing of nanomedicines from bench to GMP: A case study using protein-loaded liposomes. Int. J. Pharm. 2020, 582, 119266.
(20) Jahn, A.; Stavis, S. M.; Hong, J. S.; Vreeland, W. N.; DeVoe, D. L.; Gaitan, M. Microfluidic Mixing and the Formation of Nanoscale Lipid Vesicles. ACS Nano 2010, 4 (4), 2077-2087.
(21) Liu, Y.; Yang, G.; Hui, Y.; Ranaweera, S.; Zhao, C.-X. Microfluidic Nanoparticles for Drug Delivery. Small 2022, 18(36), 2106580.
(22) Ma, Z.; Li, B.; Peng, J.; Gao, D. Recent Development of Drug Delivery Systems through Microfluidics: From Synthesis to Evaluation. Pharmaceutics 2022, 14 (2).
(23) Maeki, M.; Uno, S.; Niwa, A.; Okada, Y.; Tokeshi, M. Microfluidic technologies and devices for lipid nanoparticle-based RNA delivery. J. Control. Release 2022, 344, 80-96.
(24) Shepherd, S. J.; Issadore, D.; Mitchell, M. J. Microfluidic formulation of nanoparticles for biomedical applications. Biomater. 2021, 274, 120826.
(25) Shepherd, S. J.; Han, X.; Mukalel, A. J.; El-Mayta, R.; Thatte, A. S.; Wu, J.; Padilla, M. S.; Alameh, M.-G.; Srikumar, N.; Lee, D.; et al. Throughput-scalable manufacturing of SARS-COV-2 mRNA lipid nanoparticle vaccines. Proc. Nat. Acad. Sci. 2023, 120(33 ), e2303567120.
(26) Hwang, Y.-H.; Um, T.; Ahn, G.-N.; Kim, D.-P.; Lee, H. Robust and scalable production of emulsion-templated microparticles in 3D-printed milli-fluidic device. Chem. Eng. J. 2022, 431, 133998.
(27) Seo, H.; Jeon, L.; Kwon, J.; Lee, H. High-Precision Synthesis of RNA-Loaded Lipid Nanoparticles for Biomedical Applications. Adv. Health. Mater. 2023, 12(13), 2203033.
(28) Cho, W. K.; Kong, B.; Choi, I. S. Highly Efficient Non-Biofouling Coating of Zwitterionic Polymers: Poly((3-(methacryloylamino)propyl)-dimethyl(3-sulfopropyl)ammonium hydroxide). Langmuir 2007, 23 (10), 5678-5682.
(29) Massenburg, S. S.; Amstad, E.; Weitz, D. A. Clogging in parallelized tapered microfluidic channels. Microfluid. Nanofluidics 2016, 20, 1-5.
(30) Wang, Z.; Voicu, D.; Tang, L.; Li, W.; Kumacheva, E. Microfluidic studies of polymer adsorption in flow. Lab Chip 2015, 15 (9), 2110-2116.
(31) Schoenitz, M.; Grundemann, L.; Augustin, W.; Scholl, S. Fouling in microstructured devices: a review. Chem. Commun. 2015, 51(39), 8213-8228,
(32) Lowe, S.; O'Brien-Simpson, N. M.; Connal, L. A. Antibiofouling polymer interfaces: poly(ethylene glycol) and other promising candidates. Poly. Chem. 2015, 6 (2), 198-212.
(33) Hu, J.; Zhang, H.; Sun, B.; Lu, A.; Zhong, G.; Chen, Z. Integration of antifouling and foul-release moieties for optimizing the performance of PEG-silicone coatings. J. Coat. Technol. Res. 2021, 18(6), 1669-1677.
(34) Skinner, M.; Johnston, B. M.; Liu, Y.; Hammer, B.; Selhorst, R.; Xenidou, I.; Perry, S. L.; Emrick, T. Synthesis of Zwitterionic Pluronic Analogs. Biomacromol. 2018, 19 (8), 3377-3389.
(35) Abraham, M. H.; Acree, W. E., Jr. Solvation Descriptors for Zwitterionic α-Aminoacids; Estimation of Water-Solvent Partition Coefficients, Solubilities, and Hydrogen-Bond Acidity and Hydrogen-Bond Basicity. ACS Omega 2019, 4 (2), 2883-2892.
(36) Lee, S.-W.; Tettey, K. E.; Kim, I. L.; Burdick, J. A.; Lee, D. Controlling the Cell-Adhesion Properties of Poly(acrylic acid)/Polyacrylamide Hydrogen-Bonded Multilayers. Macromol. 2012, 45(15), 6120-6126.
(37) Leslie, D. C.; Waterhouse, A. ; Berthet, J. B.; Valentin, T. M.; Watters, A. L.; Jain, A.; Kim, P. ; Hatton, B. D.; Nedder, A.; Donovan, K.; et al. A bioinspired omniphobic surface coating on medical devices prevents thrombosis and biofouling. Nat. Biotechnol. 2014, 32 (11), 1134-1140.
(38) Wang, X.-T.; Deng, X.; Zhang, T.-D.; Zhang, J.; Chen, L.-L.; Wang, Y.-F.; Cao, X.; Zhang, Y.-Z.; Zheng, X.; Yin, D.-C. A Versatile Hydrophilic and Antifouling Coating Based on Dopamine Modified Four-Arm Polyethylene Glycol by One-Step Synthesis Method. ACS Macro Lett. 2022, 11(6), 805-812.
(39) Epstein, A. K.; Wong, T.-S.; Belisle, R. A.; Boggs, E. M.; Aizenberg, J. Liquid-infused structured surfaces with exceptional anti-biofouling performance. Proc. Natl. Acad. Sci. 2012, 109(33 ), 13182-13187.
(40) MacCallum, N.; Howell, C.; Kim, P.; Sun, D.; Friedlander, R.; Ranisau, J.; Ahanotu, O.; Lin, J. J.; Vena, A.; Hatton, B.; et al. Liquid-Infused Silicone As a Biofouling-Free Medical Material. ACS Biomater. Sci. Eng. 2015, 1 (1), 43-51.
(41) Howell, C.; Grinthal, A., Sunny, S.; Aizenberg, M.; Aizenberg, J.
Designing Liquid-Infused Surfaces for Medical Applications: A Review. Adv. Mater. 2018, 30(50 ), 1802724.
(42) Douglass, M.; Garren, M.; Devine, R.; Mondal, A.; Handa, H. Bioinspired hemocompatible surface modifications for biomedical applications. Prog. Mater. Sci. 2022, 130, 100997.
(43) Gidi, Y.; Bayram, S.; Ablenas, C. J.; Blum, A. S.; Cosa, G. Efficient One-Step PEG-Silane Passivation of Glass Surfaces for Single-Molecule Fluorescence Studies. ACS Appl. Mater. Interfaces 2018, 10 (46), 39505-39511.
(44) Estephan, Z. G.; Jaber, J. A.; Schlenoff, J. B. Zwitterion-Stabilized Silica Nanoparticles: Toward Nonstick Nano. Langmuir 2010, 26 (22), 16884-16889.
(45) Estephan, Z. G.; Hariri, H. H.; Schlenoff, J. B. One-pot, exchange-free, room-temperature synthesis of sub-10 nm aqueous, noninteracting, and stable zwitterated iron oxide nanoparticles. Langmuir 2013, 29 (8), 2572-2579.
(46) Stroock, A. D.; Dertinger, S. K.; Ajdari, A.; Mezic, I.; Stone, H. A.; Whitesides, G. M. Chaotic mixer for microchannels. Science 2002, 295 (5555), 647-651.

(47) Romanowsky, M. B.; Abate, A. R.; Rotem, A.; Holtze, C.; Weitz, D. A. High throughput production of single core double emulsions in a parallelized microfluidic device. Lab Chip 2012, 12 (4), 802-807.
(48) Yadavali, S.; Jeong, H.-H.; Lee, D.; Issadore, D. Silicon and glass very large scale microfluidic droplet integration for terascale generation of polymer microparticles. Nat. Commun. 2018, 9 (1), 1222.
(49) Swingle, K. L.; Hamilton, A. G.; Mitchell, M. J. Lipid Nanoparticle-Mediated Delivery of mRNA Therapeutics and Vaccines. Trends. Mol. Med. 2021, 27(6), 616-617.
(50) Howell, C.; Vu, T. L.; Johnson, C. P.; Hou, X.; Ahanotu, O.; Alvarenga, J.; Leslie, D. C.; Uzun, O.; Waterhouse, A.; Kim, P.; et al. Stability of Surface-Immobilized Lubricant Interfaces under Flow. Chem. Mater. 2015, 27 (5), 1792-1800.
(51) Kolle, S.; Ahanotu, O.; Meeks, A.; Stafslien, S.; Kreder, M.; Vanderwal, L.; Cohen, L.; Waltz, G.; Lim, C. S.; Slocum, D.; et al. On the mechanism of marine fouling-prevention performance of oil-containing silicone elastomers. Sci. Rep. 2022, 12 (1), 11799.
(52) Ozkan, E.; Mondal, A.; Douglass, M.; Hopkins, S. P.; Garren, M; Devine, R.; Pandey, R., Manuel, J.; Singha, P.; Warnock, J.; et al. Bioinspired ultra-low fouling coatings on medical devices to prevent device-associated infections and thrombosis. J. Coll. Interf. Sci. 2022, 608, 1015-1024.
(53) Roberts, T. R.; Choi, J. H.; Wendorff, D. S.; Harea, G. T.; Beely, B. M.; Sieck, K. N.; Douglass, M. E.; Singha, P.; Dean, J. B.; Handa, H.; et al. Tethered Liquid Perfluorocarbon Coating for 72 Hour Heparin-Free Extracorporeal Life Support. ASAIO J. 2021, 67(7).
(54) Sunny, S.; Vogel, N.; Howell, C.; Vu, T. L.; Aizenberg, J. Lubricant-Infused Nanoparticulate Coatings Assembled by Layer-by-Layer Deposition. Adv. Funct. Mater. 2014, 24(42), 6658-6667.

### ASPECTS

The following Aspects are illustrative only and do not limit the scope of the present disclosure or the appended claims. Any part or parts of any one or more Aspects can be combined with any part or parts of any one or more other Aspects.

Aspect 1. A microfluidic component, comprising: at least one microchannel configured to communicate a fluid flow therein, the at least one microchannel comprising at least one surface having a tethered liquid perfluorocarbon (TLP) coating disposed thereon, the at least one microchannel optionally having a cross-sectional dimension in the range of from about 10 μm to about 1000 μm.

Aspect 2. The microfluidic component of Aspect 1, wherein the TLP coating comprises a liquid perfluorocarbon associated with a perfluorocarbon tethered to surface of the at least one microchannel. Some example perfluoruocarbons that can be included in a TLP coating are perfluoropropane, perfluorobutane, perfluoropentane, perfluorohexane, perfluorooctane, perfluorodecalin, perfluorooctylbromide, and perfluoroperhydrophenanthrene.

Aspect 3. The microfluidic component of any one of Aspects 1-2, wherein the at least one microchannel is defined in glass, silicon, cyclic olefin copolymer, polyvinylchloride, acrylic, or PDMS.

Aspect 4. The microfluidic component of Aspect 3, wherein the at least one microchannel is defined in glass.

Aspect 5. The microfluidic component of Aspect 3, wherein the at least one microchannel is defined in silicon.

Aspect 6. The microfluidic component of Aspect 3, wherein the at least one microchannel is defined in PDMS.

Aspect 7. The microfluidic component of any one of Aspects 1-6, wherein the at least one microchannel has a cross-sectional dimension in the range of from about 10 μm to about 1000 μm, optionally from about 10 μm to about 500 μm.

Aspect 8. The microfluidic component of any one of Aspects 1-7, wherein the at least one channel is comprised in a mixer.

Aspect 9. The microfluidic component of Aspect 8, wherein the mixer is characterized as a herringbone mixer.

Aspect 10. The microfluidic component of any one of Aspects 1-8, further comprising a fluid disposed in the at least one microchannel, the fluid comprising at least one of (1) water and a water-miscible solvent, (2) a lipid nanoparticle, (3) a surfactant, and (4) a biomolecule or a portion of a biomolecule.

Aspect 11. The microfluidic component of Aspect 10, wherein the fluid comprises water and a water-miscible solvent, the water-miscible solvent optionally comprising an alcohol.

Aspect 12. The microfluidic component of any one of Aspects 1-11, wherein the microfluidic component is comprised in a system for formulating lipid nanoparticles.

Aspect 13. A method, comprising operating a system to formulate lipid nanoparticles, the system comprising a microfluidic component according to any one of Aspects 1-12.

Aspect 14. The method of Aspect 13, wherein the system is operable to form lipid nanoparticles having a PDI of less than about 0.4 for up to about 30 minutes.

Aspect 15. The method of Aspect 14, wherein the system is operable to form lipid nanoparticles having a PDI of less than about 0.4 for up to about 50 minutes. A system can also be operable for more than 50 minutes-for example for 60 minutes-to form lipid nanoparticles having a PDI of less than about 0.4.

Aspect 16. The method of any one of Aspects 13-15, wherein the system is operable for up to about 30 minutes to form lipid nanoparticles having a PDI that varies by less than about 15%.

Aspect 17. The method of any one of Aspects 13-16, wherein the system is operable to form lipid nanoparticles having an encapsulation efficiency of above about 80% for up to about 30 minutes.

Aspect 18. The method of Aspect 17, wherein the system is operable to form lipid nanoparticles having an encapsulation efficiency of above about 80% for up to about 50 minutes.

Aspect 19. The method of Aspect 18, wherein the system is operable to form lipid nanoparticles having a PDI of less than about 0.4 for up to about 50 minutes.

Aspect 20. The method of any one of Aspects 13-19, wherein the system is operable for up to about 30 minutes to form lipid nanoparticles having a PDI that varies by less than about 15%.

1. A microfluidic component, comprising:
at least one microchannel configured to communicate a fluid flow therein,
the at least one microchannel comprising at least one surface having a tethered liquid perfluorocarbon (TLP) coating disposed thereon,
the at least one microchannel optionally having a cross-sectional dimension in the range of from about 10 μm to about 1000 μm.

2. The microfluidic component of claim 1, wherein the TLP coating comprises a liquid perfluorocarbon associated with a perfluorocarbon tethered to surface of the at least one microchannel.

3. The microfluidic component of claim 1, wherein the at least one microchannel is defined in glass, silicon, cyclic olefin copolymer, polyvinylchloride, acrylic, or PDMS.

4. The microfluidic component of claim 3, wherein the at least one microchannel is defined in glass.

5. The microfluidic component of claim 3, wherein the at least one microchannel is defined in silicon.

6. The microfluidic component of claim 3, wherein the at least one microchannel is defined in PDMS.

7. The microfluidic component of claim 1, wherein the at least one microchannel has a cross-sectional dimension in the range of from about 10 μm to about 1000 μm, optionally from about 10 μm to about 500 μm.

8. The microfluidic component of claim 1, wherein the at least one microchannel is comprised in a mixer.

9. The microfluidic component of claim 8, wherein the mixer is characterized as a herringbone mixer.

10. The microfluidic component of any claim 1, further comprising a fluid disposed in the at least one microchannel, the fluid comprising at least one of (1) water and a water-miscible solvent, (2) a lipid nanoparticle, (3) a surfactant, and (4) a biomolecule or a portion of a biomolecule.

11. The microfluidic component of claim 10, wherein the fluid comprises water and a water-miscible solvent, the water-miscible solvent optionally comprising an alcohol.

12. The microfluidic component of claim 1, wherein the microfluidic component is comprised in a system for formulating lipid nanoparticles.

13. A method, comprising operating a system to formulate lipid nanoparticles, the system comprising a microfluidic component according to claim 1.

14. The method of claim 13, wherein the system is operable to form lipid nanoparticles having a PDI of less than about 0.4 for up to about 30 minutes.

15. The method of claim 14, wherein the system is operable to form lipid nanoparticles having a PDI of less than about 0.4 for up to about 50 minutes.

16. The method of claim 13, wherein the system is operable for up to about 30 minutes to form lipid nanoparticles having a PDI that varies by less than about 15%.

17. The method of claim 13, wherein the system is operable to form lipid nanoparticles having an encapsulation efficiency of above about 80% for up to about 30 minutes.

18. The method of claim 17, wherein the system is operable to form lipid nanoparticles having an encapsulation efficiency of above about 80% for up to about 50 minutes.

19. The method of claim 18, wherein the system is operable to form lipid nanoparticles having a PDI of less than about 0.4 for up to about 50 minutes.

20. The method of claim 13, wherein the system is operable for up to about 30 minutes to form lipid nanoparticles having a PDI that varies by less than about 15%.
